(19) United States
(12) Patent Application Publication
(10) Pub. No.: US 2018/0206780 A1
(43) Pub. Date: Jul. 26, 2018

(54) Laser-Based System And Method For Diagnosing Pigmented Lesions

(71) Applicant: SPECLIPSE, INC., Seoul (KR)

(72) Inventors: Sung Hyun PYUN, Seoul (KR); Wan Ki MIN, Gyeonggi-do (KR)

(73) Assignee: SPECLIPSE, INC.

(21) Appl. No.: 15/895,509

(22) Filed: Feb. 13, 2018

Related U.S. Application Data
(63) Continuation of application No. PCT/KR2018/001074, filed on Jan. 24, 2018.

(30) Foreign Application Priority Data
Jan. 26, 2017 (KR) 10-2017-0012935
Mar. 10, 2017 (KR) 10-2017-0030624

(51) Int. Cl. A61B 5/00

(52) U.S. Cl. CPC A61B 5/444; A61B 5/7435; A61B 5/0095

(57) ABSTRACT

According to one embodiment of the present disclosure, a device for diagnosing a pigmented lesion includes: a sound detector configured to detect a photoacoustic wave generated when a laser is irradiated onto a target from which a pigmented lesion is to be identified; and a diagnosis unit configured to calculate a depth and/or thickness of the pigmented lesion, based on a time at which the photoacoustic wave is detected, and laser irradiation time information on the laser irradiated onto the target.

### CROSS REFERENCE TO RELATED APPLICATIONS

The present application is a continuation of PCT/KR2018/001074 filed 24 Jan. 2018, which claims priority to Korean Patent Application Nos. 10-2017-0030624 filed on 10 Mar. 2017 and 10-2017-0012935 filed 26 Jan. 2017 in the Republic of Korea, the disclosures of which are incorporated herein by reference.

### FIELD

The present disclosure relates to a device and a method for diagnosing a pigmented lesion, and a handpiece and a handpiece detachable device used therein.

The present disclosure was supported by National Research and Development Project Business as follows:

[Project Number] S2365593

[Related Department] Small & Medium Business Administration

[Research Management Specialized Agency] Korea Business Angels Association

[Research Business Name] Global Market Type Startup Business R&D (TIPS program)

[Research Project Title] Real-time, non-invasive, In-vivo cancer diagnosis technology utilizing lasers

[Contribution Rate] 1/1

[Main Institute] Speclipse, Inc.

[Research Period] Oct. 1, 2015-Sep. 30, 2017

### BACKGROUND

Pigmented lesions are one of skin diseases and may be divided into benign lesions and malignant lesions. For example, the benign lesion may include freckle or nevus of Ota, and the malignant lesion may include a skin cancer.

The benign lesion may be removed with lasers by using a skin toning device or a skin peeling device, and the wavelength of a laser to be used varies according to the location of a benign lesion (depth from a skin).

In related-art methods, it is difficult to distinguish between benign lesions and malignant lesions. Therefore, many people who engage in removing pigmented lesions with lasers by using a skin toning device or a skin peeling device may treat pigmented lesions without exactly distinguishing them. Even if they can distinguish between benign lesions and malignant lesions, they should really cut out benign lesions or use expensive equipment, such as an Optical Coherence Tomography (OCT), in order to measure depths of benign lesions. This may cause inconvenience.

### SUMMARY

### Technical Objects

According to one embodiment of the present disclosure, there are provided a device and a method for diagnosing a pigmented lesion, which can determine a depth and/or thickness of a pigmented lesion.

According to another embodiment of the present disclosure, there are provided a device and a method for diagnosing a pigmented lesion, which can recommend a type of laser of a wavelength which can remove a benign lesion to a user, based on a depth and/or thickness of a pigmented lesion, and can inform the user of a development state and a degree of seriousness of a malignant lesion.

According to still another embodiment of the present disclosure, there are provided a handpiece and a handpiece detachable device used for the above-described devices and methods for diagnosing the pigmented lesion.

### Technical Solving Means

According to one embodiment of the present disclosure, there is provided a device for diagnosing a pigmented lesion, the device including: a sound detector configured to detect a photoacoustic wave generated when a laser is irradiated onto a target from which a pigmented lesion is to be identified; and a diagnosis unit configured to calculate a depth of the pigmented lesion, based on a time at which the photoacoustic wave is detected, and laser irradiation time information on the laser irradiated onto the target.

According to another embodiment of the present disclosure, there is provided a method for diagnosing a pigmented lesion, the method including: irradiating a laser onto a target from which a pigmented lesion is to be identified; detecting a photoacoustic wave generated from the target onto which the laser is irradiated; and calculating a depth and/or thickness of the pigmented lesion, based on a time at which the photoacoustic wave is detected and laser irradiation time information on the laser irradiated onto the target.

According to still another embodiment of the present disclosure, there is provided a system for diagnosing a pigmented lesion, the system including: a laser generation device configured to irradiate a laser onto a target from which a pigmented lesion is to be identified; a diagnosis device configured to detect a photoacoustic wave generated when the laser is irradiated onto the pigmented lesion, and to select a type of a laser for treating the pigmented lesion based on the detected photoacoustic wave; and a laser controller configured to control a laser wavelength, power, and/or a pulse width according to the type of the laser selected by the diagnosis device.

### Advantageous Effect

According to one or more embodiments of the present disclosure, the device and the method may recommend a type of a laser having a wavelength capable of removing a benign lesion to a user based on a depth and/or thickness of a pigmented lesion, and may inform the user of a development state of a malignant lesion. In addition, according to one or more embodiments of the present disclosure, the device and the method may diagnose whether a pigmented lesion is a benign lesion or a malignant lesion, measure the depth and/or thickness of the pigmented lesion, recommend a type of a laser capable of removing the benign lesion when the pigmented lesion is determined to be the benign lesion, and inform the user of a development state of the malignant lesion when the pigmented lesion is determined to be the malignant lesion.

The handpiece according to one or more embodiments of the present disclosure may be usefully used in the devices and the methods for diagnosing the pigmented lesion, which achieve the above-described effects.

### BRIEF DESCRIPTION OF THE DRAWINGS

FIG. 1 is a view to illustrate a device for diagnosing a pigmented lesion according to one embodiment of the present disclosure;

FIG. 2 is a view to illustrate a device for diagnosing a pigmented lesion according to another embodiment of the present disclosure;

FIG. 3 is a view to illustrate a device for diagnosing a pigmented lesion according to still another embodiment of the present disclosure;

FIG. 4A and FIG. 4B are views to illustrate a principle based on which a device for diagnosing a pigmented lesion calculates a location of a pigmented lesion according to one embodiment of the present disclosure;

FIGS. 5 and 6 are views to illustrate an example in which a device for diagnosing a pigmented lesion is mounted on a handpiece according to one embodiment of the present disclosure;

FIG. 7 is a view to illustrate a method for measuring a thickness of a pigmented lesion according to one embodiment of the present disclosure;

FIGS. 8 and 9 are views to illustrate a handpiece according to one embodiment of the present disclosure;

FIG. 10 is a view to illustrate a handpiece according to another embodiment of the present disclosure;

FIG. 11A and FIG. 11B are views to illustrate a handpiece according to still another embodiment of the present disclosure;

FIG. 12A and FIG. 12B are views to illustrate a handpiece according to yet another embodiment of the present disclosure;

FIGS. 13 and 14 are views to illustrate a handpiece according to further embodiment of the present disclosure; and

FIG. 15 is a view to illustrate a method for diagnosing a pigmented lesion according to one embodiment of the present disclosure.

[Explanation of Signs]

| | |
|---|---|
| 1, 201, 301, 401: laser | 3, 203, 303: handpiece |
| 5, 205, 405: sound detector | 7, 207, 407: diagnosis unit |
| 206: detector | 209, 409: light detector |
| 302: laser controller | 433, 733: first piece |
| 443, 743: second piece or | |
| handpiece detachable device | |
| 418: second body portion | 414: light detection module |
| 416: sound detection module | 422: guide portion |

### DETAILED DESCRIPTION

Exemplary embodiments will now be described more fully with reference to the accompanying drawings to clarify aspects, other aspects, features and advantages of the present disclosure. The exemplary embodiments may, however, be embodied in many different forms and should not be construed as limited to the exemplary embodiments set forth herein. Rather, the exemplary embodiments are provided so that this disclosure will be thorough and complete, and will fully convey the scope of the application to those of ordinary skill in the art.

If the terms such as “first” and “second” are used to describe elements, these elements should not be limited by such terms. These terms are used for the purpose of distinguishing one element from another element only. The exemplary embodiments include their complementary embodiments.

In the description of this application, the term “element A and/or element B” is used to refer to “at least one of element A and element B.”

In the description of this application, when “element A” and “element B” are referred to as being coupled to each other, “element A” and “element B” are directly coupled to each other or indirectly coupled to each other. Herein, indirectly coupling means that there is one or more third elements between “element A” and “element B.”

The terms “unit” and “module” and the terms having suffix “-er” or “-or” used in the description of this application refer to a unit for processing at least one function or operation, and may be implemented by hardware, software, or a combination of hardware and software.

Expressions such as “transmitting,” “communicating,” “receiving,” “providing,” or “forwarding” signals, data, or information, used in the description of this application, or other expressions similar to the aforementioned expressions may refer to directly forwarding signals, data, or information from one element (“element a”) to another element (“element b”), and also refer to forwarding to element b via at least one other element (“element c”).

In the description of this application, elements “operatively related to each other” should be interpreted as being connected with each other in a wired and/or wireless manner so as to transmit and/or receive data.

Although there is no explicit expression “an element (“element a”) and another element (“element b”) are operatively related to each other” in the description, if element a receives signals, data, or information outputted from element b and performs its operation (“element a”), or if element b receives signals, data, or information outputted from element a and performs its operation (“element b”), it should be understood that element a and element b are “operatively related to each other.”

In the description of this application, a “laser generation device” refers to a device which generates a laser for beauty or medical care, and an “aesthetic or medical laser handpiece” refers to a device which has a shape to be gripped by a user with user's hand and is configured to receive a laser generated by the laser generation device and emit the laser to a target.

In the description of this application, the term “laser” means a pulse laser or a continuous light laser. In addition, the frequency band of the “laser” may have a certain frequency band, for example, an ultra violet (UV) band, a visible light band, or an infrared (IR) band.

In the description of this application, the term “generated light” encompasses all types of light which are generated when a laser is irradiated onto a target (for example, body tissue). Accordingly, the “generated light” may refer to plasma light, reflected light, scattered light, and/or fluorescent light.

In the description of this application, the term “wavelength” refers to a specific numerical value or a numerical value of a specific range (that is, a wavelength band).

In the description of this application, the “handpiece” may be formed of one piece, two pieces, or three or more pieces. In the description of this application, when the handpiece is formed of two pieces, one piece will be referred to as a first piece, and the other piece will be referred to as a second piece. Herein, the second piece is detachably coupled to the first piece and may be referred to as a “handpiece detachable device.”

In the description of this application, the term “light path” refers to a path through which a laser travels, and may be a space or a wire or fiber that is formed of a material that can make it easy for the laser to travel.

The terms used herein are for the purpose of describing particular exemplary embodiments only and are not intended to limit the present disclosure. As used herein, the singular forms are intended to include the plural forms as well, unless the context clearly indicates otherwise. It will be further understood that the terms “comprises” and/or “comprising,” when used in this specification, do not preclude the presence or addition of one or more other components.

Hereinafter, the present disclosure will be described in greater detail with reference to the accompanying drawings. In describing specific embodiments, various specific features are described to assist in a detailed description and a comprehensive understanding of the present disclosure. However, it is apparent that the exemplary embodiments can be carried out by those of ordinary skill in the art without those specifically defined features. In the description of exemplary embodiments, certain detailed explanations of portions which are well known and have nothing to do with the present disclosure are omitted when it is deemed that they may unnecessarily obscure the essence of the present disclosure.

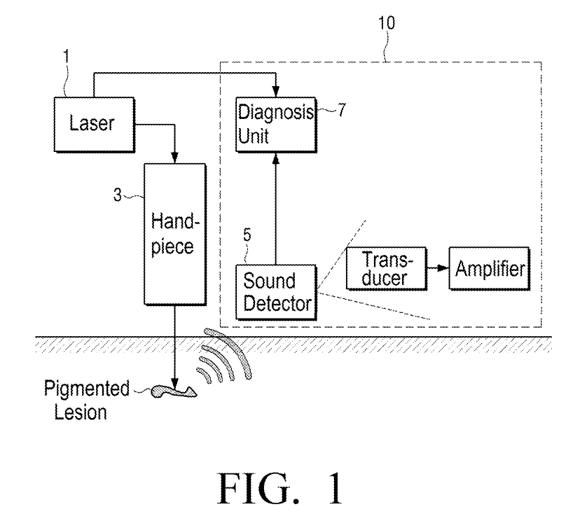

FIG. 1 is a view to illustrate a device for diagnosing a pigmented lesion according to one embodiment of the present disclosure.

Referring to FIG. 1, a system for diagnosing a pigmented lesion according to one embodiment of the present disclosure includes a laser generation device 1, an aesthetic or medical laser handpiece 3 (hereinafter, referred to as a “handpiece”), and a device 10 for diagnosing a pigmented lesion (hereinafter, referred to as a “diagnosis device”). The diagnosis device 10 may include a sound detector 5 and a diagnosis unit 7.

The diagnosis device 10 may further include a circuit means (not shown) for providing a result of sound detection of the sound detector 5 to the diagnosis unit 7 although it is not illustrated. Herein, the circuit means (not shown) may be configured to provide the result of detection to the diagnosis unit 7 in a wire or wireless manner.

For example, a wire cable (not shown) may be provided in the handpiece 3 to provide the result of detection detected by the sound detector 5 to the diagnosis unit 7. The wire cable may have one end connected with the sound detector 5 and the other end connected with the diagnosis unit 7. Such a wire cable may be equally applied to other embodiments which will be described below.

In the description of this application, the “result of sound detection” may be used to refer to a “sound detection time” or “data for calculating the sound detection time.”

In the present embodiment, the sound detector 5 may detect a sound which is generated when a laser is irradiated onto a target (for example, a skin) from which a depth and/or thickness of a pigmented lesion is to be identified. For example, the sound detector 5 may include a transducer (not shown) to convert a sound into an electric signal to detect a sound, and an amplifier (not shown) to amplify the electric signal converted by the transducer.

The sound detector 5 may be coupled to a body portion of the handpiece 3 to be brought into contact with the target when the handpiece 3 is brought into contact with the target and a laser is irradiated to diagnose the depth and/or thickness of the pigmented lesion. When the handpiece 3 is formed of two pieces, the sound detector 5 may be coupled to a lower portion of a body portion of the second piece as will be described below.

When the laser generated by the laser generation device 1 is irradiated onto the target through the handpiece 3, the pigmented lesion existing in the target may absorb the laser and generate a sound. This phenomenon is referred to as a photoacoustic phenomenon, and the sound generated by the photoacoustic phenomenon will be referred to as a “photoacoustic wave” in the following description.

In the present embodiment, the diagnosis unit 7 may calculate the depth of the pigmented lesion based on a time at which the photoacoustic wave is detected by the sound detector 5 (“sound detection time”), and laser irradiation time information.

In the description of this application, the term “laser irradiation time information” may be used to mean at least one of:

i) a laser generation time at which the laser generation device generates a laser to be irradiated onto a target;

ii) a time at which the laser reaches the surface of the target;

iii) a time at which light generated from the surface of the target after the laser is irradiated onto the target is detected; and

iv) a certain time between the laser generation time and the time at which the generated light is detected.

The sound detector 5 may detect the photoacoustic wave which is generated from the target, and may provide the result of sound detection to the diagnosis unit 7.

The sound detector 5 may detect one or more photoacoustic waves, and the diagnosis unit 7 may calculate the depth and/or thickness of the pigmented lesion using the sound detection time and the laser irradiation time information.

In the present embodiment, the diagnosis unit 7 may receive the laser generation time from the laser generation device 1, and receive the result of sound detection from the sound detector 5, and may calculate the depth and/or thickness of the pigmented lesion.

The sound detector 5 may detect at least two photoacoustic waves.

In the description of this application, a wave that is detected first in time is referred to as a first photoacoustic wave, and a wave which is detected next is referred to as a second photoacoustic wave for convenience of explanation. When the first photoacoustic wave and the second photoacoustic wave satisfy the following condition, the first photoacoustic wave and the second photoacoustic wave are referred to as a “photoacoustic wave couple.”

Condition: in which the first photoacoustic wave and the second photoacoustic wave are detected after the laser is irradiated onto the target, and a time at which the first photoacoustic wave is detected and a time at which the second photoacoustic wave is detected are different from each other, and it is determined that the first photoacoustic wave is generated when the laser is absorbed onto the top surface of the pigmented lesion (a surface close to the surface of the target), and the second photoacoustic wave is generated when the laser is absorbed onto the bottom surface of the pigmented lesion (a surface far from the surface of the target).

The diagnosis unit 7 may calculate the thickness of the pigmented lesion based on a result of detecting the photoacoustic wave couple.

The diagnosis unit 7 may perform an operation of selecting one or more types of lasers for treating the pigmented lesion with reference to a laser type DB when the pigmented lesion is a benign lesion. In the description of this application, the term “laser type” is data defining a laser and refers to data defining a laser according to at least one of a wavelength, power, a pulse width, and a product name.

Specifically, the diagnosis unit 7 selects one or more types of lasers corresponding to the depth and/or thickness of the pigmented lesion from the laser type DB.

For example, the diagnosis unit 7 may select one or more types of lasers in consideration of the depth of the pigmented lesion, or may select one or more types of lasers in consideration of both the depth and the thickness of the pigmented lesion.

This is because the wavelength, power, and width (duration) of the laser for removing or reducing the pigmented lesion vary according to the depth and/or thickness of the pigmented lesion. Although not shown, the laser type DB (data defining types of lasers according to depths and/or thicknesses of pigmented lesions) may be prepared, and the diagnosis unit 70 may select a type of a laser according to the depth and/or thickness of the pigmented lesion with reference to the laser type DB.

When the pigmented lesion is a malignant lesion, the diagnosis unit 7 may determine a development state of the malignant lesion with reference to the pigmented lesion DB (not shown). Herein, the malignant lesion may be a skin cancer, for example, and the pigmented lesion DB (not shown) may be data which defines a development state according to the depth and/or thickness of the malignant lesion.

As described above, when the pigmented lesion is a malignant lesion, the diagnosis unit 7 may determine the development state of the malignant lesion according to the depth and/or thickness of the pigmented lesion with reference to the pigmented lesion DB (not shown). For example, the diagnosis unit 7 may determine the development state of the pigmented lesion according to the thickness of the pigmented lesion, or may determine the development state of the pigmented lesion by considering both the depth and the thickness of the pigmented lesion.

“Data defining a development state” may indicate a development stage of a cancer (first stage, second stage, third stage, fourth stage).

The above-described pigmented lesion DB and the laser type DB may be included in the diagnosis device 10 or may be separately stored in an external storage device that the diagnosis unit 7 can access in a wire or wireless manner. When the pigmented lesion DB and the laser type DB are stored in the external storage device, the diagnosis device 10 may access the pigmented lesion DB and the laser type DB stored in the external storage device and may refer to data.

Although not shown, a display like a monitor may display a result of an operation of the diagnosis unit 7. For example, a type of a laser selected by the diagnosis unit or the development state of the pigmented lesion may be displayed on the display (not shown).

In the above-described embodiment, the diagnosis unit 7 performs the operation of selecting the type of the laser when the pigmented lesion is a benign lesion, and performs the operation of determining the development state when the pigmented lesion is a malignant lesion. In an alternative embodiment, the diagnosis unit 7 may be configured to perform both two operations regardless of the type of the pigmented lesion. That is, in an alternative embodiment, the diagnosis unit 7 may determine the type of the laser and the development state based on the depth and/or thickness of the pigmented lesion regardless of the type of the pigmented lesion.

In the above-described embodiments, a device for determining the type of the pigmented lesion has not been mentioned. However, a device for determining the type of the pigmented lesion may be separately prepared or the diagnosis unit 7 may be configured to additionally include the function of determining the type of the pigmented lesion. For example, the device (not shown) for determining the type of the pigmented lesion may be configured to determine whether the pigmented lesion is a malignant lesion by analyzing a spectrum of generated light generated from the pigmented lesion when a laser is irradiated onto the pigmented lesion. In another example, the device (not shown) for determining the type of the pigmented lesion may be configured to determine whether the pigmented lesion is a malignant lesion by analyzing the spectrum of the generated light generated from the pigmented lesion and an image of the pigmented lesion. Korean Patent Registration No. 10-1640202 (Jul. 11, 2016) discloses technology of determining whether a pigmented lesion is a malignant lesion by analyzing a spectrum of generated light generated from the pigmented lesion and/or an image of the pigmented lesion. The entirety of features disclosed in Korean Patent Registration No. 10-1640202 (Jul. 11, 2016) is incorporated herein as a part of the description of this application.

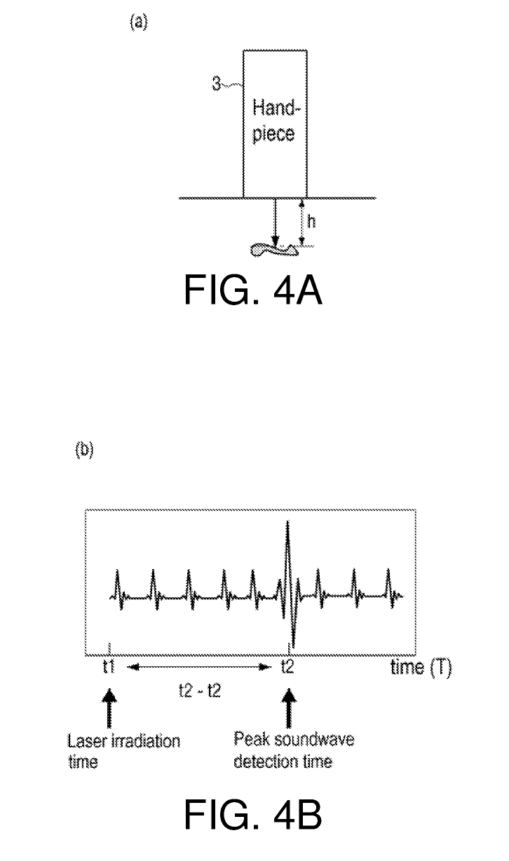

FIG. 4A and FIG. 4B are views to illustrate a principle for calculating a depth (location) of a pigmented lesion in the pigmented lesion diagnosis device according to one embodiment of the present disclosure.

Hereinafter, a method in which the diagnosis unit 7 calculates a depth (or location) of a pigmented lesion will be described with reference to FIGS. 1 and 4.

In the description of this application, a depth (or location) of a pigmented lesion is defined by a distance (h) from the surface of a target to the pigmented lesion (for example, the surface lesion (top surface, bottom surface or side surface) of the pigmented lesion or a certain location in the pigmented lesion) (see FIG. 4A). In FIG. 4A, the depth (h) of the pigmented lesion is illustrated by a distance to the center of the pigmented lesion, but this is merely an example.

For example, the diagnosis unit 7 may calculate the depth of the pigmented lesion based on following Equation 1:

Depth (h) of a pigmented lesion=speed of a photoacoustic wave×(sound detection time−laser irradiation time) Equation 1

The “laser irradiation time” included in Equation 1 refers to one of:

i) a laser generation time at which the laser generation device generates a laser to be irradiated onto a target;

ii) a time at which the laser reaches the surface of the target;

iii) a time at which light generated from the surface of the target after the laser is irradiated onto the target is detected; and

iv) a certain time between the laser generation time and the time at which the generated light is detected.

Herein, the sound detection time refers to a time at which a photoacoustic wave generated from the pigmented lesion is detected. When a pulse laser is irradiated onto the pigmented lesion, a photoacoustic wave having a relatively higher peak than in the other portions may be generated from the pigmented lesion as shown in FIG. 4B.

In addition, the diagnosis unit 7 may calculate the thickness of the pigmented lesion based on following Equation 2:

Thickness (d) of a pigmented lesion=speed of a photoacoustic wave×(second photoacoustic wave detection time−first photoacoustic wave detection time) Equation 2

Herein, the first photoacoustic wave and the second photoacoustic wave are a photoacoustic wave couple.

In the present embodiment, the entirety or a portion of the elements included in the pigmented lesion diagnosis device 10 may be configured to be coupled to the handpiece 3.

In the present embodiment, the handpiece 3 may be configured to include a body portion which has an inner space to receive a laser and to output the laser to the outside, and is formed in a cylindrical shape to be easily held by user's hand.

In the present embodiment, the handpiece 3 may be formed of one piece, two pieces, or three or more pieces.

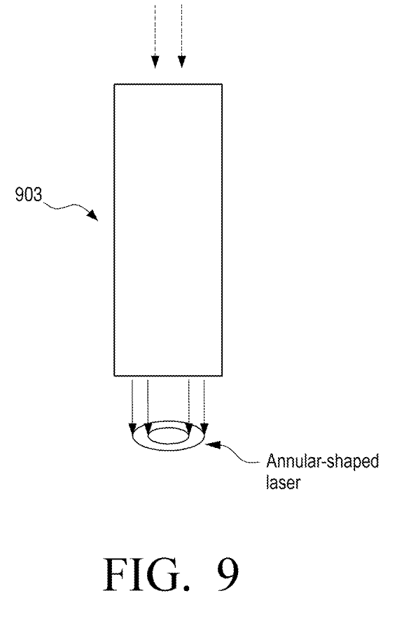

When the handpiece 3 is formed of one piece, the handpiece 3 may be configured to include a cylindrical body portion having an inner space to receive a laser and to output the laser to the outside, and an upper portion of the body portion may be an input end to receive the laser from the outside, and a lower portion of the body portion may be an output end to output the received laser to the outside (target). When the handpiece 3 is formed of one piece, the sound detector 5 may be disposed outside or inside the body portion. For example, the sound detector 5 may be coupled to the output end. Specifically, the sound detector 5 may be coupled to the output end to be bright into contact with the target when the pigmented lesion is diagnosed. Descriptions of exemplary embodiments when the handpiece 3 is formed of one piece may be replaced with descriptions of other drawings (for example, FIGS. 9, 10, 11, and 12) which will be provided below.

When the handpiece 3 is formed of two pieces, one piece is referred to as a first piece and the other piece is referred to as a second piece. Herein, the second piece may be detachably coupled to the first piece.

The first piece may be configured to include a cylindrical body portion (first body portion) having an inner space to receive a laser and to output the laser to the outside, and the second piece may be configured to include a cylindrical body portion (second body portion) having an inner space to receive a laser and to output the laser to the outside. The first body portion and the second body portion may be detachably coupled to each other.

An upper portion of the first body portion may be an input end to receive the laser from the outside, and a lower portion of the first body portion may be an output end to output the laser entering through the input end to the outside. In addition, an upper portion of the second body portion may be detachably coupled to the lower portion of the first body portion to serve as an input end to receive the laser outputted from the first body portion, and a lower portion of the second body portion may be an output end to output the received laser to the outside (target).

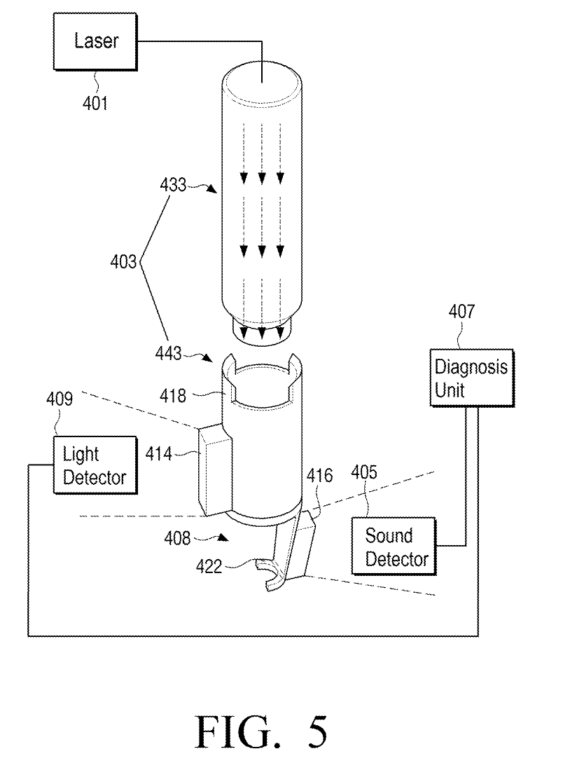

When the handpiece 3 is formed of two pieces, the sound detector 5 may be disposed outside or inside the second piece (that is, a handpiece detachable device). For example, the sound detector 5 may be coupled to the output end of the second piece. Specifically, the sound detector 5 may be coupled to the output end of the second piece to be brought into contact with the target when the pigmented lesion is diagnosed. Descriptions of exemplary embodiments when the handpiece 3 is formed of two pieces may be replaced with descriptions of other drawings (for example, FIGS. 5, 6, 9, 10, 13, 14), which will be provided below.

The above-described configuration of the handpiece 3 is equally applied to embodiments which will be described below.

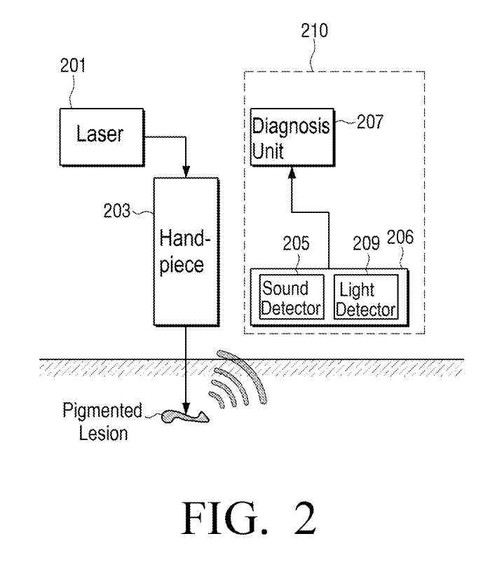

FIG. 2 is a view to illustrate a device for diagnosing a pigmented lesion according to one embodiment of the present disclosure.

Referring to FIG. 2, a system for diagnosing a pigmented lesion according to one embodiment of the present disclosure may include a laser generation device 201, a handpiece 203, and a device 210 for diagnosing a pigmented lesion according to one embodiment of the present disclosure, and the diagnosis device 210 may include a detector 206 and a diagnosis unit 207.

From among the elements of the embodiment of FIG. 2 and the elements of the embodiment of FIG. 1, elements given similar reference numerals perform the same or similar functions. Accordingly, hereinafter, the difference of the embodiment of FIG. 2 from the embodiment of FIG. 1 will be highlighted.

In the present embodiment, the detector 206 may detect a photoacoustic wave and a laser. To achieve this, the detector 206 may include a sound detector 205 and a light detector 209.

The sound detector 205 may detect a photoacoustic wave which is generated when a laser is irradiated onto a target (for example, a skin) from which a depth and/or thickness of a pigmented lesion is to be identified.

The sound detector 205 may be coupled to the body portion of the handpiece 203 to be brought into contact with the target when the handpiece 203 is brought into contact with the target and the laser is irradiated onto the target to diagnose the depth and/or thickness of the pigmented lesion. When the handpiece 203 is formed of two pieces, the sound detector 205 may be coupled to the lower portion of the body portion of the second piece.

The light detector 209 may detect the laser irradiated onto the target through the handpiece 203 or generated light that is generated from the target when the laser is irradiated onto the target.

The light detector 209 may be implemented by a device such as a photodiode, for example. The light detector 209 may be configured to detect the laser traveling through the inside of the handpiece 203, to detect the laser discharged from the handpiece 203 and traveling to the target, or to detect the generated light generated from the target.

For example, when the light detector 209 is configured to detect the laser traveling through the inside of the handpiece 203, the light detector 209 may be disposed inside or outside the handpiece 203. In this case, an optical element (not shown in FIG. 2) may be disposed inside the handpiece 203 to split at least a portion of the laser traveling through the inside of the handpiece 203 toward the light detector 209.

When the light detector 209 is configured to detect the laser discharged from the handpiece 203 and traveling to the target, or to detect the generated light generated from the target, the light detector 209 may be disposed adjacent to a specific portion (a portion to be brought into contact with the target) of the handpiece 203, or may be disposed in a handpiece detachable device (not shown in FIG. 2) which is detachably coupled to the handpiece 203.

The diagnosis unit 207 may calculate the depth and/or thickness of the pigmented lesion, based on a time at which the photoacoustic wave is detected by the sound detector 205, and the result of laser detection of the light detector 209. The “result of laser detection” may include “laser irradiation time information.”

In addition, the diagnosis unit 207 may select a type of a laser and/or a development state based on the depth and/or thickness of the pigmented lesion.

The diagnosis device 210 may further include a circuit means (not shown) for providing the result of detection of the detector 209 to the diagnosis unit 207 although it is not illustrated in FIG. 2. Herein, the circuit means (not shown) may be configured to provide the result of detection to the diagnosis unit 207 in a wire or wireless manner.

For example, a wire cable (not shown) may be provided in the handpiece 203 to provide the result of detection detected by the detector 206 to the diagnosis unit 207. The wire cable may be configured to have one end connected with the detector 206 and the other end connected to the diagnosis unit 207.

The diagnosis unit 207 may receive the result of sound detection and the result of laser detection from the detector 206, and may calculate the depth and/or thickness of the pigmented lesion based on the results of detection.

For example, the diagnosis unit 207 may calculate the depth (h) of the pigmented lesions by using Equation 1 described above, and may calculate the thickness (d) of the pigmented lesion by using Equation 2 described above.

For example, the diagnosis unit 207 may determine a type of a laser according to the depth (h) and/or thickness of the pigmented lesion. The type of the laser according to the depth (d) and/or thickness of the pigmented lesion may be displayed through a display such as a monitor (not shown) connected with the diagnosis unit 207 in a wire or wireless manner although it is not illustrated. As described above, the type of the laser may be data that is defined by at least one of a wavelength, power, a pulse width, and a product name.

In the present embodiment, the entirety or a portion of the elements included in the pigmented lesion diagnosis device 210 may be configured to be detachably coupled to the handpiece 203. For example, the detector 206 may be detachably coupled to the handpiece 203, and the diagnosis unit 207 may be connected with the detector 205 in a wire or wireless manner to receive the result of sound detection and the result of laser detection.

In the present embodiment, the handpiece 203 may be configured to include a body portion which has an inner space to receive a laser and to output the laser to the outside and is formed in a cylindrical shape to be gripped by user's hand. The body portion may be formed of one piece, two pieces, or three or more pieces.

In the present embodiment, the handpiece 203 may be configured to include a cylindrical body portion having an inner space to receive a laser and to output the laser to the outside.

In the present embodiment, the handpiece 203 may be formed of one piece, two pieces, or three or more pieces.

When the handpiece 203 is formed of one piece, the handpiece 203 may be configured to include one cylindrical body portion having an inner space to receive the laser and to output the laser to the outside, and the detector 206 may be disposed outside or inside the body portion. Descriptions of exemplary embodiments when the handpiece 3 is formed one piece may be replaced with descriptions of other drawings (for example, FIGS. 9, 10, 11, and 12).

When the handpiece 203 is formed of two pieces, the detector 206 may be disposed outside or inside the second piece (that is, a handpiece detachable device). Descriptions of exemplary embodiments when the handpiece is formed of two pieces may be replaced with descriptions of other drawings (for example, FIGS. 5, 6, 9, 10, 13, and 14).

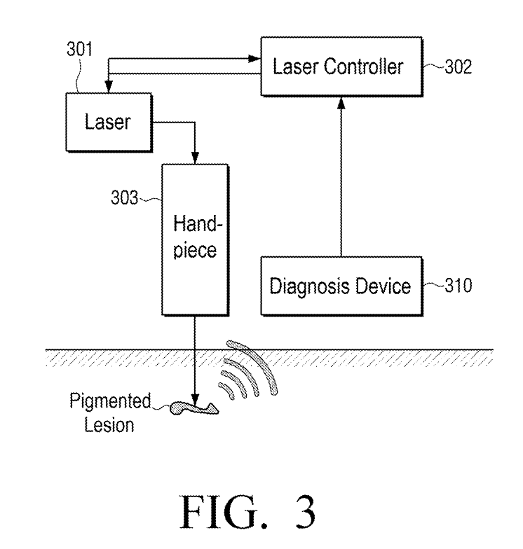

FIG. 3 is a view to illustrate a device for diagnosing a pigmented lesion according to one embodiment of the present disclosure.

Referring to FIG. 3, a system for diagnosing a pigmented lesion according to one embodiment of the present disclosure may include a laser generation device 301, a laser controller 302, a handpiece 303, and a device 310 for diagnosing a pigmented lesion according to one embodiment of the present disclosure. The diagnosis device 310 may be the pigmented lesion diagnosis device 10 described with reference to FIG. 1, or the pigmented lesion diagnosis device 210 described with reference to FIG. 2.

The pigmented lesion diagnosis device 310 according to the present embodiment may perform the same or similar function as or to that of the pigmented lesion diagnosis device 10 described with reference to FIG. 1, or the pigmented lesion diagnosis device 210 described with reference to FIG. 2. Accordingly, hereinafter, the difference of the embodiment of FIG. 3 from the embodiments of FIG. 1 or 2 will be highlighted.

In the present embodiment, when a laser generated by the laser generation device 301 is irradiated onto a target from which a pigmented lesion is to be identified, the diagnosis device 310 may detect a photoacoustic wave generated when the laser is irradiated onto the pigmented lesion, and may select a type of a laser for treating the pigmented lesion based on the detected photoacoustic wave, and the laser controller 302 may control an operation of the laser generation device 301 according to the type of the laser selected by the diagnosis device 310. That is, the laser controller 301 controls the laser generation device 301 to generate a laser having a laser wavelength, power, and/or a pulse width which is defined by the type of the laser provided by the diagnosis device 310.

The pigmented lesion diagnosis device 310 may calculate a depth and/or thickness of the pigmented lesion based on a sound detection time and laser irradiation time information, and may select a type of a laser for treating the pigmented lesion having such a depth and/or thickness.

The pigmented lesion diagnosis device 310 may inform the laser controller 320 of the type of the laser selected as described above.

The laser controller 302 may control the operation of the laser generation device 301 to generate a laser having a wavelength, power, and/or a pulse width defined according to the type of the laser provided from the pigmented lesion diagnosis device 310.

The laser generation device 301 may generate a laser according to the wavelength, power, and/or pulse width determined under the control of the laser controller 302.

In the present embodiment, the entirety or a portion of the elements included in the pigmented lesion diagnosis device 310 may be configured to be detachably coupled to the handpiece 303.

In the present embodiment, the handpiece 303 may have the same configuration as that of the handpiece described with reference to FIG. 1 or 2.

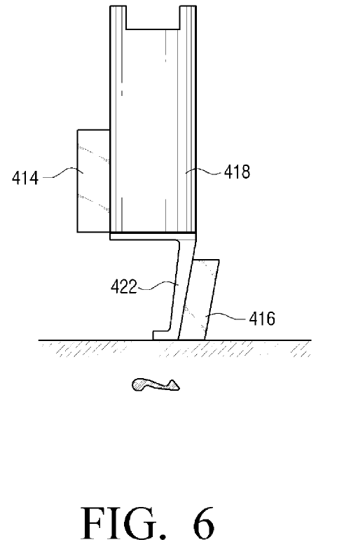

FIGS. 5 and 6 are views to illustrate an example of a pigmented lesion diagnosis device mounted on a handpiece according to one embodiment of the present disclosure.

Referring to FIGS. 5 and 6, the pigmented lesion diagnosis device according to one embodiment of the present disclosure may include a sound detector 405, a light detector 409, and a diagnosis unit 407. In the present embodiment, the sound detector 405 and the light detector 409 may be disposed on the handpiece 403.

In the present embodiment, the handpiece 403 may be configured to include two pieces, that is, a first piece 433 and a second piece 443. The second piece 443 may be detachably coupled to the first piece 433.

In the present embodiment, the first piece 433 may include a cylindrical body portion (first body portion) having an inner space to receive a laser and to output the laser to the outside, and the second piece 443 may include a cylindrical body portion 418 (second body portion) having an inner space to receive the laser discharged from the first piece 433 and to output the laser to a target. In the present embodiment, since the first piece 433 itself is the first body portion 433, the first piece 433 and the first body portion 433 are interchangeably used without being distinguished from each other.

In the present embodiment, the second piece 443, that is, a handpiece detachable device 443, may be detachably coupled to the first piece 433. In the present embodiment, the sound detector 405 and the light detector 409 may be coupled to a lower portion of the body portion 418 of the handpiece detachable device 443.

In the present embodiment, the functions of the sound detector 405 and the diagnosis unit 407 are the same or similar as or to the functions of the sound detector and the diagnosis unit described with reference to FIG. 1, 2, or 3, and the function of the light detector 409 is the same as the function of the light detector described with reference to FIG. 2 or 3, and thus a detailed description of the sound detector 405, the diagnosis unit 407, or the light detector 409 will be omitted.

The pigmented lesion diagnosis device may additionally include a circuit means (not shown) to provide the result of detection of the sound detector 405 to the diagnosis unit 407 although it is not illustrated in FIGS. 5 and 6. Herein, the circuit means (not shown) may be configured to provide the result of detection to the diagnosis unit 407 in a wire or wireless manner.

For example, a wire cable (not shown) may be provided in the detachable device 443 to provide the result of detection detected by the sound detector 405 to the diagnosis unit 407, and the wire cable may have one end connected with the sound detector 405 and the other end connected with the diagnosis unit 407.

Accordingly, the difference of the embodiment of FIG. 5 from the embodiments of FIGS. 1 to 3 will be highlighted.

The handpiece detachable device 443 may be detachably coupled to the first piece 433.

In the present embodiment, the handpiece detachable device 443 may include the second body portion 418, a guide portion 422, a light detection module 414, and a sound detection module 416.

In the present embodiment, the light detection module 414 may be coupled to the second body portion 418, and the sound detection module 416 may be coupled to the guide portion 422.

The second body portion 418 has a structure that is detachably coupled to the first piece 433. That is, the second body portion 418 may be coupled to the first piece 433 by a user's force, and the coupling state may be maintained as long as there is no external force. The second body portion 418 and the first piece 433 may be decoupled from each other by an external force.

The second body portion 418 may be formed in a cylindrical shape to have a space formed therein to allow a laser to pass therethrough. According to the present embodiment, the second body portion 418 may be formed in a cylindrical shape to have a space formed therein to allow a laser to pass therethrough, and may have an end to receive the laser from the handpiece 433 (hereinafter, an “input end”), and an end to discharge the laser to the target (hereinafter, an “output end”). Herein, the input end has a structure to be detachably coupled to the first piece 433, and the output end is coupled to the guide portion 422.

In the present embodiment, the light detection module 414 is attached to an outside of the second body portion 418 to detect a laser irradiated onto the target, and the sound detection module 416 may be attached to the guide portion 422 to detect a sound generated from the pigmented lesion. In the present embodiment, as shown in FIG. 6, the sound detection module 416 is brought into contact with the target along with an end of the guide portion 422 (a portion brought into contact with the target). That is, an end (a portion brought into contact with the target) of the sound detection module 416, and the end (portion brought into contact with the target) of the guide portion 422 may be aligned with each other. This is to directly bring the sound detector 405 included in the sound detection module 416 into contact with the target when the guide portion 422 is brought into contact with the target to diagnose the pigmented lesion on the skin.

The light detection module 414 includes the light detector 409. The light detector 409 may detect the laser irradiated onto the target, and may provide the result of laser detection to the diagnosis unit 407.

The sound detection module 416 includes the sound detector 405. The sound detector 405 may detect a photoacoustic wave generated from the pigmented lesion, and may provide the result of sound detection to the diagnosis unit 407.

The diagnosis unit 407 may calculate the depth and/or thickness of the pigmented lesion based on the result of sound detection and the result of laser detection. The diagnosis unit 407 may calculate the depth (h) of the pigmented lesion by using Equation 1 described above, and may calculate the thickness (d) of the pigmented lesion by using Equation 2 described above.

The diagnosis unit 407 may select a type of a laser according to the depth (h) and/or thickness of the pigmented lesion. As described above, the laser type DB defines a laser wavelength, laser power, a laser width, and/or a laser product name according to the depth (d) and/or thickness of the pigmented lesion, and the diagnosis unit 407 may select the type of the laser according to the depth (h) and/or thickness of the pigmented lesion by referring to the laser type DB.

The wavelength of the laser for treating the pigmented lesion varies according to the location of the pigmented lesion. For example, a Q-switched Ruby laser (694 nm) and a Q-switched Alexandrite laser (755 nm) may treat a pigmented lesion existing on the top of epidermis, and a Q-switched Nd:YAG laser (532 nm) may treat a pigmented lesion existing in epidermis, and a Q-switched Nd:YAG laser (1064 nm) may treat a pigmented lesion existing in corium.

In the present embodiment, a display such as a monitor connected with the diagnosis unit 407 in a wire or wireless manner may display the type of the laser according to the depth (d) and/or thickness (d) of the pigmented lesion, and accordingly, the user may easily know the type of the laser for treating the pigmented lesion.

Although the handpiece 403 is formed of two pieces in the embodiment of FIG. 5, the handpiece 403 may be formed of one piece. For example, the first piece 433 and the second piece 443 may be fixedly coupled to each other.

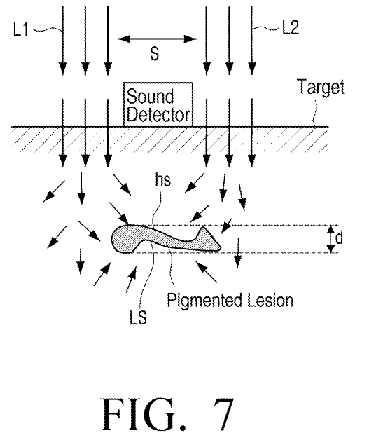

FIG. 7 is a view to illustrate a photoacoustic wave couple generated when lasers L1, L2 spaced apart from each other by a predetermined distance (s) are irradiated onto the pigmented lesion of the target.

A first photoacoustic wave may be generated when the laser is absorbed onto a top surface (hs) of the pigmented lesion, and a second photoacoustic wave may be generated when the laser is absorbed onto a bottom surface (Ls) of the pigmented lesion, and, when these photoacoustic waves have a photoacoustic wave couple relationship, the thickness (d) of the pigmented lesion may be calculated.

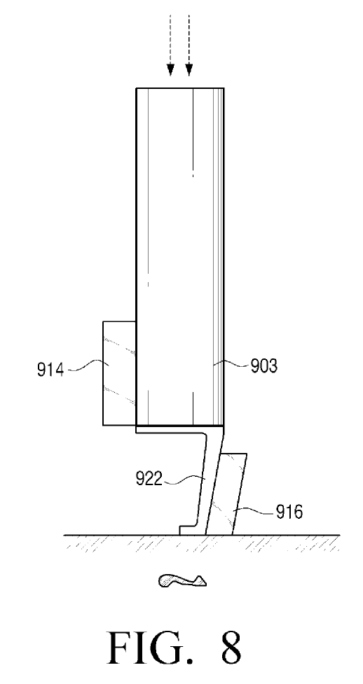

In order to generate the second photoacoustic wave when the laser is absorbed onto the bottom surface (Ls) of the pigmented lesion, the laser may be irradiated onto the periphery of the pigmented lesion rather than onto the center of the pigmented lesion. Methods for irradiating the laser onto the periphery of the pigmented lesion (preferably, so as to surround the pigmented lesion) may include a method for irradiating an annular-shaped laser as shown in FIGS. 8 to 11.

In the description of this application, the “annular-shaped” laser refers to a laser of a certain shape that is irradiated so as to surround an entirety or a portion of the periphery of the pigmented lesion rather than being directly irradiated onto the pigmented lesion.

FIGS. 8 and 9 are views to illustrate a handpiece according to one embodiment of the present disclosure.

Referring to FIGS. 8 and 9, the handpiece 903 according to one embodiment of the present disclosure may irradiate an annular-shaped laser. That is, the handpiece 903 may receive a laser (not shown) of a certain shape and may irradiate the annular-shaped laser to surround a pigmented lesion.

In the present embodiment, the handpiece 903 may be formed of one piece, two pieces, or three or more pieces. When the handpiece 903 is formed of two pieces, the second piece may be detachably coupled to the first piece.

The handpiece 903 may include a sound detector 916, and descriptions of a location and a function of the sound detector 916 may be replaced with descriptions of FIGS. 1, 2, 3, and 5.

In addition, the handpiece 903 may further include a light detector 914, and, descriptions of a location and a function of the light detector 914 may be replaced with descriptions of FIGS. 2, 3, and 5.

In addition, the light detector 914 and the sound detector 916 may be connected with a diagnosis unit (not shown), and a description of a function of the diagnosis unit may be replaced with descriptions of FIGS. 1, 2, 3, and 5.

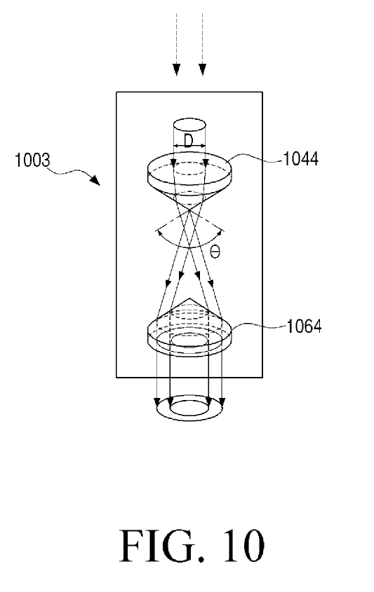

According to the present embodiment, the handpiece 903 may output the annular-shaped laser to surround the pigmented lesion, and exemplary configurations regarding this will be described below with reference to FIGS. 10 to 14.

FIG. 10 is a view to illustrate a handpiece according to another embodiment of the present disclosure.

Referring to FIG. 10, the handpiece 1003 according to an embodiment of the present disclosure may irradiate an annular-shaped laser. That is, the handpiece 1003 may receive a laser of a certain shape (not shown) and may irradiate the received laser in an annular shape to surround a pigmented lesion.

According to the present embodiment, the handpiece 1003 may include at least two optical elements, and the at least two optical elements may be optically coupled to each other so as to irradiate the annular-shaped laser.

In the present embodiment, the handpiece 1003 may be formed of one piece, two pieces, or three or more pieces. When the handpiece 1003 is formed of two pieces, the second piece may be detachably coupled to the first piece.

Referring to FIG. 10, the at least two optical elements included in the handpiece 1003 may be a first optical element 1044 and a second optical element 1064 which are of a conical shape. The first optical element 1044 and the second optical element 1064 each may include a conical portion and a flat portion.

In the present embodiment, the first optical element 1044 and the second optical element 1064 may be arranged so as to allow the laser entering the body portion of the handpiece 1003 to pass through the first optical element 1044 and the second optical element 1064 in sequence.

For example, the laser entering the body portion of the handpiece 1003 may enter the flat portion of the first optical element 1044 and may be discharged through the conical portion. In addition, the laser discharged through the conical portion of the first optical element 1044 may enter the conical portion of the second optical element 1064 and may be discharged through the flat portion of the second optical element 1064. That is, the first optical element 1044 and the second optical element 1064 may be optically coupled to each other so as to irradiate the annular-shaped laser.

For convenience of explanation, the other elements such as the light detector, the sound detector, and the guide portion of FIG. 10 are not illustrated, but these elements may be included in the handpiece 1003.

That is, the handpiece 1003 may include a sound detector (for example, the sound detector described with reference to FIG. 1, 2, 3, or 5), and descriptions of a location and a function of the sound detector may be replaced with descriptions of FIGS. 1, 2, 3, and 5.

In addition, the handpiece 1003 may further include a light detector (for example, the light detector described with reference to FIG. 2, 3, or 5), and descriptions of a location and a function of the light detector may be replaced with descriptions of FIGS. 2, 3, and 5.

In addition, the handpiece 1003 may be connected with a diagnosis unit (for example, the diagnosis unit described with reference to FIG. 1, 2, 3, or 5), and a description of a function of the diagnosis unit may be replaced with descriptions of FIGS. 1, 2, 3, and 5.

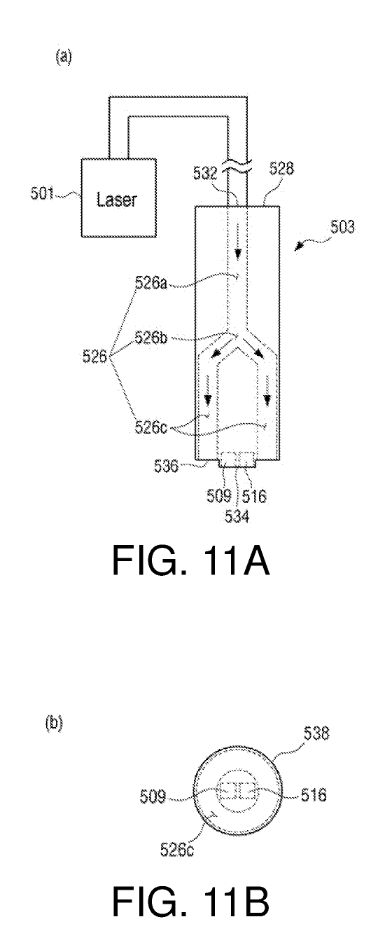

FIG. 11A and FIG. 11B are views to illustrate a handpiece according to still another embodiment of the present disclosure.

Referring to FIGS. 11A and 11B, the handpiece 503 according to one embodiment of the present disclosure may irradiate an annular-shaped laser. That is, the handpiece 503 may receive a laser of a certain shape from a laser generation device 501, and may irradiate the received laser in an annular shape to surround a pigmented lesion.

The handpiece 503 according to the present embodiment may be formed of one piece.

The handpiece 503 may include a cylindrical body portion to be gripped by user's hand, which has an inner space to receive a laser from the outside and to output the laser to the outside. The body portion may include a first end portion 528, a second end portion 534, and a light path 526.

The second end portion 534 is a portion that is brought into contact with a target. In the present embodiment, the second end portion 534 may be configured to have a center portion protruding therefrom.

The first end portion 528 may have an input end 532 formed on a certain region thereof to receive the laser from the laser generation device, and the second end portion 534 may have an output end 536 formed on a certain region thereof to discharge the laser traveling through the light path to the target.

The light path 526 may be disposed in the cylindrical body portion, and may be configured to guide the laser entering through the input end 532 to be discharged through the output end 536.

According to the present embodiment, the light path 526 may include a first light path 526a, a light split portion 526b, and a second light path 526c. The first light path 526a is a path that guides the laser entering through the input end 532, and the light split portion 526b splits the laser toward the second light path 526c.

The second light path 526c may be formed to allow the laser to be irradiated onto the target in a circular ring shape. In order to irradiate the laser in the circular ring shape, the light split portion 526b may allow the laser entering through the input end 532 and traveling through the first light path 526a to travel toward an outer edge 538 of the second end portion 534, and the output end 536 may be formed along the outer edge of the second end portion.

That is, in the present embodiment, the light path 526 may be configured to allow the laser entering in a straight line shape to be irradiated in the circular ring shape.

The light path 526 illustrated in FIGS. 11A and 11B is an example, and the light path 526 may be configured to include two or more light split portions, differently from that of FIGS. 11A and 11B, or may be configured to irradiate the laser in other ring shapes (for example, an oval ring shape or a semicircular ring shape).

In the embodiment of FIGS. 11A and 11B, the first light path 526a and the light split portion 526b may be directly connected with each other, and the light split portion 526b and the second light path 526c may be directly connected with each other. However, this is merely an example and the first light path 526a, the light split portion 526b, and the second light path 526c may be differently configured.

In another example, one or more other light paths may be formed between the first light path 526a and the light split portion 526b, and the one or more other light paths may perform a function of guiding the laser from the first light path 526a to the light split portion 526b.

In addition, the handpiece 503 may be configured to further include a sound detector 516 to detect a photoacoustic wave, and a light detector 509 to detect the laser.

The sound detector 516 may be coupled to a lower portion of the body portion of the handpiece to be brought into contact with the target when the handpiece 503 is brought into contact with the target and the laser is irradiated in order to diagnose the depth and/or thickness of the pigmented lesion.

In the present embodiment, as shown in FIGS. 11A and 11B, the sound detector 516 may be disposed on a certain region (a portion not overlapping the output end) of the second end portion 534. In the present embodiment, regions of the sound detector 516 and the output end 536 are defined so as to prevent the laser from passing through the region of the sound detector 516, but the output end 536 may be disposed to surround the sound detector 516 (that is, in an annular shape).

As shown in FIGS. 11A and 11B, the light detector 509 may be disposed on a certain region (a portion not overlapping the output end 536 or the sound detector 516) of the second end portion 534. In the present embodiment, regions of the light detector 509 and the output end 536 are defined so as to prevent the laser from passing through the region of the light detector 509, but the output end 536 may be disposed to surround the light detector 509 (that is, in an annular shape).

As described above, the handpiece 503 is configured to output the laser in the annular shape, such that a photoacoustic wave couple can be effectively generated.

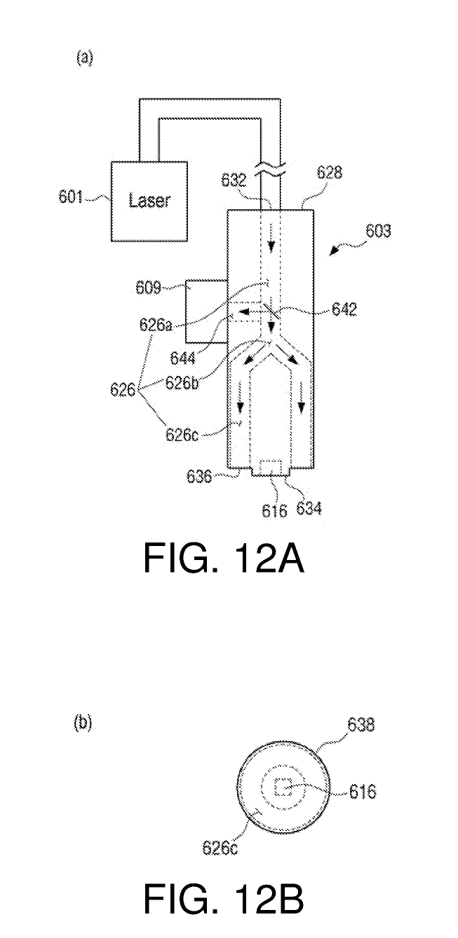

FIG. 12A and FIG. 12B are views to illustrate a handpiece according to yet another embodiment of the present disclosure.

Referring to FIGS. 12A and 12B, the handpiece according to yet another embodiment of the present disclosure may be used as a portion of the above-described pigmented lesion diagnosis device.

Referring to FIGS. 12A and 12B, the handpiece 603 may irradiate an annular-shaped laser. That is, the handpiece 603 may receive a laser of a certain shape from a laser generation device 601, and may irradiate the received laser in an annular shape to surround a pigmented lesion.

The handpiece 603 according to the present embodiment may be formed of one piece.

The handpiece 603 may include a cylindrical body portion to be gripped by user's hand, which has an inner space to receive a laser from the outside and to output the laser to the outside. The body portion may include a first end portion 628, a second end portion 634, and a light path 626.

From among the elements of the embodiment of FIGS. 12A and 12B and the elements of the embodiment of FIGS. 11A and 11B, elements given similar reference numerals may perform the same or similar functions. Accordingly, hereinafter, the difference of the embodiment of FIGS. 12A and 12B from the embodiment of FIGS. 11A and 11B will be highlighted.

Referring to FIGS. 12A and 12B, the handpiece 603 may be configured to include the cylindrical body portion to be gripped by user's hand, and the cylindrical body portion may include the first end portion 628, the second end portion 634, and the light path 626.

Each of the first end portion 628 and the second end portion 634 may be a portion of the cylindrical shape. In the present embodiment, the second end portion 634 may be a portion that is brought into contact with a target, and may be configured to have a center portion protruding therefrom.

The first end portion 628 may have an input end 632 formed on a certain region thereof to receive the laser from the laser generation device 601, and the second end portion 634 may have an output end 636 formed on a certain region thereof to discharge the laser traveling through the light path to the target.

The light path 626 may be disposed in the cylindrical body portion, and may be configured to guide the laser entering through the input end 632 to be discharged through the output end 636.

According to the present embodiment, the light path 626 may include a first light path 626a, a light split portion 626b, and a second light path 626c. The first light path 626a is a path that guides the laser entering through the input end 632, and the light split portion 626b splits the laser toward the second light path 626c.

The second light path 626c may be formed to allow the laser to be irradiated onto the target in a circular ring shape. In order to irradiate the laser in the circular ring shape, the light split portion 626b may allow the laser entering through the input end 632 and traveling through the first light path 626a to travel toward an outer edge 638 of the second end portion 634, and the output end 636 may be formed along the outer edge 638 of the second end portion 634.

In addition, the handpiece 603 according to the present embodiment may include a sound detector 616 to detect a photoacoustic wave, and a light detector 609 to detect the laser.

The sound detector 616 may be coupled to a lower portion of the body portion to be brought into contact with the target when the handpiece 603 is brought into contact with the target and the laser is irradiated in order to diagnose the depth and/or thickness of the pigmented lesion.

The light detector 609 may be configured and disposed to receive a portion of the laser traveling through the light path in the handpiece 603. To achieve this, the handpiece 603 may further include an optical element 642 to split a portion of the laser traveling through the light path 626 in the handpiece 603 toward the light detector 609.

In the present embodiment, the light detector 609 detects a portion of the laser guided by the first light path 626a. However, this is merely an example, and the light detector 609 may be disposed to detect a portion of the laser split by other paths, for example, by the light split portion 626b, or to detect the laser guided by the second light path 626c.

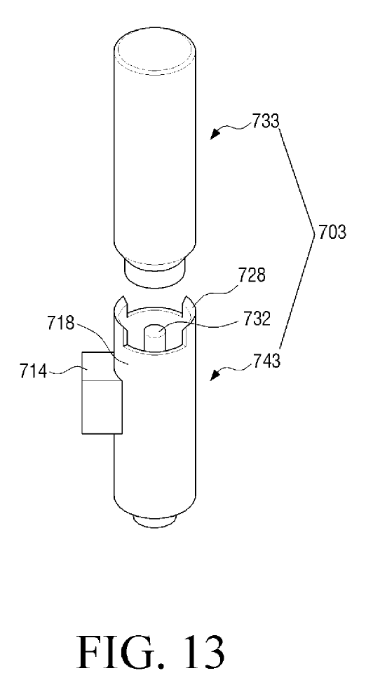

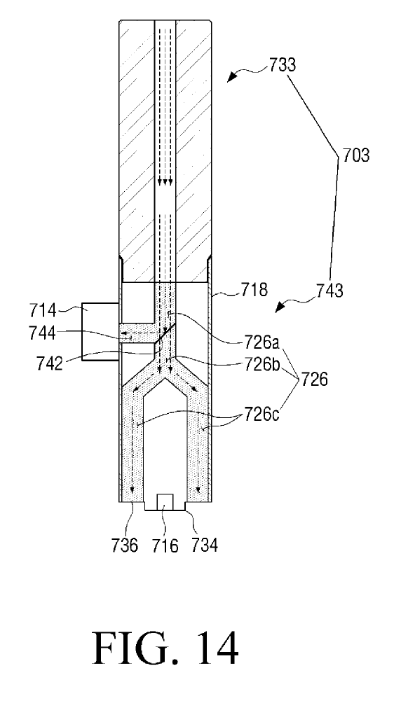

FIGS. 13 and 14 are views to illustrate a handpiece according to further embodiment of the present disclosure.

Referring to these drawings, a pigmented lesion diagnosis device according to the present embodiment may include a sound detection module 716, a light detection module 714, and a diagnosis unit (not shown). In the present embodiment, the sound detection module 716 and the light detection module 714 may be disposed on the handpiece 703. The sound detection module 716 may include a sound detector, and the light detection module 714 may include a light detector. Descriptions of the functions of the sound detector and the light detector may be replaced with the descriptions of FIGS. 1, 2, 3, 5, and 11.

The embodiment described with reference to FIGS. 13 and 14 is a variation of the embodiment of FIG. 5. That is, in the embodiment described with reference to FIGS. 13 and 14, the handpiece does not include the guide portion 422 and may be configured to irradiate the laser of the annular shape onto the pigmented lesion.

Hereinafter, the difference from the embodiment of FIG. 5 will be highlighted.

The handpiece 703 may irradiate an annular-shaped laser. That is, the handpiece 703 may receive a laser of a certain shape from a laser generation device (not shown), and may irradiate the received laser in an annular shape to surround a pigmented lesion.

In the present embodiment, the handpiece 703 may be configured to include two pieces, that is, a first piece 733 and a second piece 743. The second piece 743 may be detachably coupled to the first piece 733.

In the present embodiment, the first piece 733 may include a cylindrical body portion (first body portion) having an inner space to receive a laser and to output the laser to the outside, and the second piece 743 may include a cylindrical body portion 718 (second body portion) having an inner space to receive the laser discharged from the first piece 733 and to output the laser to a target. In the present embodiment, since the first piece 733 itself is the first body portion, the first piece 733 and the first body portion are interchangeably used without being distinguished from each other.

In the present embodiment, the second piece 743, that is, a handpiece detachable device 743, may be detachably coupled to the first piece 733.

The handpiece detachable device 743 may be configured to include the second body portion 718, and the second body portion 718 may include a first end portion 728, a second end portion 734, and a light path 726.

Each of the first end portion 728 and the second end portion 734 may be a portion of the cylindrical shape. In the present embodiment, the second end portion 734 may be a portion that is brought into contact with the target, and may be configured to have a center portion protruding therefrom.

The first end portion 728 may have an input end 732 formed on a certain region thereof to receive the laser from the laser generation device, and the second end portion 734 may have an output end 736 formed on a certain region thereof to discharge the laser traveling through the light path to the target.

The light path 726 may be disposed in the cylindrical body portion, and may be configured to guide the laser entering through the input end 732 to be discharged through the output end 736.

According to the present embodiment, the light path 726 may include a first light path 726a, a light split portion 726b, and a second light path 726c. The first light path 726a is a path that guides the laser entering through the input end 732, and the light split portion 726b splits the laser toward the second light path 726c.

An optical element 742 may be additionally disposed in the light path 726 to split a portion of the laser toward the light detection module 714, and the laser split by the optical element 742 may be provided to the light detection module 714 through the light path 744.

The second light path 726c may be formed to allow the laser to be irradiated onto the target in a circular ring shape. In order to irradiate the laser in the circular ring shape, the light split portion 726b may allow the laser entering through the input end 732 and traveling through the first light path 726a to be split toward an outer edge of the second end portion 734, and the output end 736 may be formed along the outer edge of the second end portion 734.

That is, in the present embodiment, the light path 726 may be configured to allow the laser entering in a straight line shape to be irradiated in the circular ring shape.

The light path 726 illustrated in FIG. 14 is an example, and the light path 726 may be configured to include two or more light split portions, differently from that of FIG. 14, or may be configured to irradiate the laser in other ring shapes (for example, an oval ring shape or a semicircular ring shape).

The sound detection module 716 including the sound detector may be coupled to a lower portion of the second body portion 718 to be brought into contact with the target when the handpiece 703 is brought into contact with the target and the laser is irradiated in order to diagnose the depth and/or thickness of the pigmented lesion.

For example, as shown in FIG. 14, the sound detection module 716 including the sound detector may be disposed on a certain region (a portion not overlapping the output end) of the second end portion 734. In the present embodiment, regions of the sound detection module 716 and the output end 736 are defined so as to prevent the laser from passing through the region of the sound detection module 716, but the output end 736 may be disposed to surround the sound detection module 716 (that is, in an annular shape).

As shown in FIG. 14, the light detection module 714 including the light detector may be coupled to an outer surface of the second body portion 718 to receive the laser traveling through the light path 744.

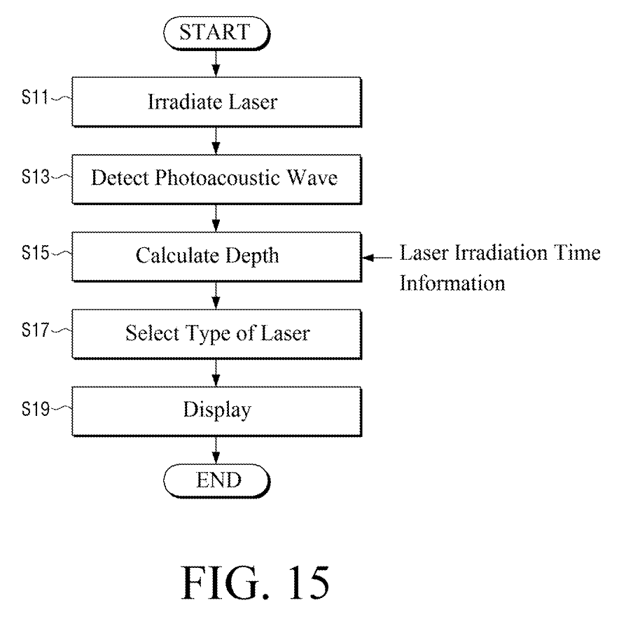

FIG. 15 is a view to illustrate a method for diagnosing a pigmented lesion according to one embodiment of the present disclosure.

Referring to FIG. 15, the method for diagnosing the pigmented lesion according to one embodiment of the present disclosure includes the steps of: irradiating a laser onto a target from which a pigmented lesion is to be identified (S11); detecting a photoacoustic wave generated from the target onto which the laser is irradiated (S13); calculating a depth and/or thickness of the pigmented lesion, based on a time at which the photoacoustic wave is detected and laser irradiation time information on the laser irradiated onto the target (S15); selecting a type of a laser based on the depth and/or thickness of the pigmented lesion calculated in step S15, and determining a development state based on the depth and/or thickness of the pigmented lesion (S17), and displaying the type of the laser and/or the development state for a user.

In step S15, the depth of the pigmented lesion may be calculated by using Equation 1 described above, and the depth of the pigmented lesion may be calculated by using Equation 2.

The method for diagnosing the pigmented lesion described with reference to FIG. 15 may be implemented by the above-described pigmented lesion diagnosis devices.

For example, the step of detecting the photoacoustic wave (S13) may be performed by the sound detector. Specifically, the step of detecting the sound (S13) may be performed by the sound detector coupled to the handpiece receiving the laser and discharging the laser to the target.

In the present embodiment, the method may further include a light detection step of detecting the laser irradiated onto the target. For example, the light detection step may be performed by the light detector which detects the laser irradiated onto the target.

For example, in step S15, the depth and/or thickness of the pigmented lesion may be calculated based on a sound detection time obtained by the sound detector, and a light detection time obtained by the light detector.

In the present embodiment, the method may further include a step of controlling a wavelength, power, and/or a pulse width of a laser generation device which generates the laser to be irradiated onto the target. For example, the step of controlling may be performed by the controller which controls the laser generation device to generate a laser having a wavelength, power, and/or a pulse width corresponding to the depth of the pigmented lesion.

While the invention has been shown and described with reference to certain preferred embodiments thereof and the drawings, the present disclosure is not limited to the above-described embodiments, and various modifications or other embodiments which belong to the equivalents of the scope of the present disclosure can be achieved by a person skilled in the art.

What is claimed is:

1. A device for diagnosing a pigmented lesion, the device comprising:
a sound detector configured to detect a photoacoustic wave generated when a laser is irradiated onto a target from which a pigmented lesion is to be identified; and
a diagnosis unit configured to calculate a depth of the pigmented lesion, based on a time at which the photoacoustic wave is detected, and laser irradiation time information on the laser irradiated onto the target.

2. The device of claim 1, wherein the sound detector is configured to detect a photoacoustic wave couple, and
wherein the diagnosis unit is configured to calculate a thickness of the pigmented lesion, based on a time at which the photoacoustic wave couple is detected, and the laser irradiation time information on the laser irradiated onto the target.

3. The device of claim 1, wherein the diagnosis unit is configured to perform an operation of selecting one or more types of lasers for treating the pigmented lesion from a laser type DB in which at least one type of laser matches a depth of the pigmented lesion, and
wherein the operation of selecting is an operation of selecting one or more types of lasers corresponding to the depth of the pigmented lesion from the laser type DB.

4. The device of claim 2, wherein the diagnosis unit is configured to determine a development state of the pigmented lesion based on the thickness of the pigmented lesion.

5. The device of claim 3, further comprising a display configured to display the type of the laser selected by the diagnosis unit for a user.

6. The device of claim 4, further comprising a display configured to display the development state of the pigmented lesion for a user.

7. The device of claim 1, further comprising a light detector configured to detect the laser irradiated onto the target,
wherein the laser irradiation time information comprises a time at which the laser is detected by the light detector.

8. The device of claim 1, wherein the sound detector is disposed on a handpiece which receives a laser generated by a laser generation device and discharges the laser to the target.

9. The device of claim 1, wherein the sound detector is disposed on a handpiece detachable device which is detachably coupled to a handpiece which receives a laser generated by a laser generation device and discharges the laser to the target.

10. The device of claim 7, wherein the light detector and the sound detector are disposed on a handpiece which receives a laser generated by a laser generation device and discharges the laser to the target.

11. The device of claim 7, wherein the light detector and the sound detector are disposed on a handpiece detachable device which is detachably coupled to a handpiece which receives a laser generated by a laser generation device and discharges the laser to the target.

12. The device of claim 1, wherein the laser irradiation time information comprises at least one of: i) a laser generation time at which a laser generation device generates a laser to be irradiated onto the target; ii) a time at which a laser reaches a surface of the target; iii) a time at which light generated from the surface of the target after a laser is irradiated onto the target is detected; and iv) a certain time between the laser generation time and the time at which the generated light is detected.

13. A method for diagnosing a pigmented lesion, the method comprising:
irradiating a laser onto a target from which a pigmented lesion is to be identified;
detecting a photoacoustic wave generated from the target onto which the laser is irradiated; and
calculating a depth and/or thickness of the pigmented lesion, based on a time at which the photoacoustic wave is detected and laser irradiation time information on the laser irradiated onto the target.

14. The method of claim 13, further comprising selecting one or more types of lasers corresponding to the depth of the pigmented lesion based on the thickness of the pigmented lesion, and determining a development state of the pigmented lesion based on the thickness of the pigmented lesion.

15. The method of claim 14, further comprising displaying the type of the laser and the development state of the pigmented lesion for a user.

16. The method of claim 14, further comprising a light detection step of detecting, by a light detector, the laser irradiated onto the target,
wherein the depth and/or thickness of the pigmented lesion is calculated based on a time at which the photoacoustic wave is detected, and a time at which light is detected by the light detector.

17. The method of claim 13, wherein the laser irradiation time information comprises at least one of: i) a laser generation time at which a laser generation device generates a laser to be irradiated onto the target; ii) a time at which a laser reaches a surface of the target; iii) a time at which light generated from the surface of the target after a laser is irradiated onto the target is detected; and iv) a certain time between the laser generation time and the time at which the generated light is detected.

18. A system for diagnosing a pigmented lesion, the system comprising:
a laser generation device configured to irradiate a laser onto a target from which a pigmented lesion is to be identified;
a diagnosis device configured to detect a photoacoustic wave generated when the laser is irradiated onto the pigmented lesion, and to select a type of a laser for treating the pigmented lesion based on the detected photoacoustic wave; and
a laser controller configured to control a laser wavelength, power, and/or a pulse width according to the type of the laser selected by the diagnosis device.

19. The system of claim 18, wherein the diagnosis device has a configuration according to claim 1.

20. The system of claim 18, wherein the diagnosis device comprises:
a sound detector configured to detect a photoacoustic wave generated when a laser is irradiated onto a target from which a pigmented lesion is to be identified; and
a diagnosis unit configured to calculate a depth of the pigmented lesion, based on a time at which the photoacoustic wave is detected, and laser irradiation time information on the laser irradiated onto the target,
wherein the sound detector is configured to detect a photoacoustic wave couple,
wherein the diagnosis unit is configured to calculate a thickness of the pigmented lesion, based on a time at which the photoacoustic wave couple is detected, and the laser irradiation time information on the laser irradiated onto the target,
wherein the diagnosis unit is configured to perform an operation of selecting one or more types of lasers for treating the pigmented lesion from a laser type DB in which at least one type of laser matches a depth of the pigmented lesion, the operation of selecting being an operation of selecting one or more types of lasers corresponding to the depth of the pigmented lesion from the laser type DB, and
wherein the laser irradiation time information comprises at least one of: i) a laser generation time at which a laser generation device generates a laser to be irradiated onto the target; ii) a time at which a laser reaches a surface of the target; iii) a time at which light generated from the surface of the target after a laser is irradiated onto the target is detected; and iv) a certain time between the laser generation time and the time at which the generated light is detected.
